(19) United States
(12) Patent Application Publication
(10) Pub. No.: US 2004/0204646 A1
(43) Pub. Date: Oct. 14, 2004

(54) Intracorporeal-imaging head

(75) Inventors: Michael Nagler, Tel Aviv (IL); Yoel Zilberstein, Haifa (IL)

Correspondence Address: Martin Moynihan, c/o ANTHONY CASTORINA, SUITE 207, 2001 JEFFERSON DAVIS HIGHWAY, ARLINGTON, VA, 22202, US

(73) Assignee: V-Target Technologies Ltd.

(21) Appl. No.: 10/836,223

(22) Filed: May 3, 2004

Related U.S. Application Data
(63) Continuation-in-part of application No. PCT/IL03/00917, filed on Nov. 4, 2003.
(60) Provisional application No. 60/423,359, filed on Nov. 4, 2002.

(51) Int. Cl. A61B 5/05; A61B 6/00

(52) U.S. Cl. 600/424; 600/436

(57) ABSTRACT

An intracorporeal-imaging head, is provided, which combines at least optical and radioactive-emission imaging, possibly also with high-resolution position tracking. The radioactive-emission-imaging probe has a wide-aperture, or coarse collimator, for high count-rate efficiency; nevertheless, the high-resolution position tracking ensures high resolution of the radioactive-emission image. Specifically, wide-aperture collimation-deconvolution algorithms are provided, for obtaining a high-efficiency, high resolution image of a radioactive-emission source, by scanning the radioactive-emission source with a probe of a wide-aperture collimator, and at the same time, monitoring the position of the radioactive-emission probe, at very fine time intervals, to obtain the equivalence of fine-aperture collimation. The blurring effect of the wide aperture is then corrected mathematically. The intracorporeal-imaging head may further include ultrasound and MRI imagers, as well as a surgical instrument, such as a biopsy needle, a knife, a cryosurgery device, a resection wire, a laser ablation device, an ultrasound ablation device, other devices for localized radiation ablations, devices for implanting brachytherapy seeds, and other minimally invasive devices. According to another embodiment, an intracorporeal-detecting head is provided, which combines at least optical and radioactive-emission detectors, for a “Yes or No” type detection, by the at least two modalities.

### CROSS-REFERENCE TO RELATED APPLICATIONS

This is a Continuation-In-Part of PCT/IL03/00917, filed on Nov. 4, 2003, which claimed priority from U.S. Provisonal application 60/423,359, filed on Nov. 4, 2002. Additionally, this application claims priority from U.S. patent application Ser. Nos. 10/616,301 and 10/616,307, both filed on Jul. 10, 2003 and from U.S. patent application Ser. No. 10/686,536, filed on Oct. 16, 2003.

### FIELD AND BACKGROUND OF THE INVENTION

The present invention relates to an intracorporeal-imaging head, and more particularly, to an intracorporeal-imaging head, which combines at least optical and gamma imaging, possibly also with high-resolution position tracking.

Radionuclide imaging is one of the most important applications of radioactivity in medicine. The purpose of radionuclide imaging is to obtain a distribution image of a radioactively labeled substance, e.g., a radiopharmaceutical, within the body following administration thereof to a patient. Examples of radiopharmaceuticals include monoclonal antibodies, such as CEA Scan (arcitumomab), made by Immunomedics Inc., or other agents, e.g., fibrinogen or fluorodeoxyglucose, tagged with a radioactive isotope, e.g., ^{99M}technetium, ⁶⁷gallium, ²⁰¹thallium, ¹¹¹indium, ¹²³iodine, ¹²⁵iodine and ¹⁸fluorine, which may be administered orally or intravenously. The radiopharmaceuticals concentrate in the area of a tumor and other pathologies such as an inflammation, since the uptake of such radiopharmaceuticals in the active part of a tumor or other pathologies is higher and more rapid than in a healthy tissue. Thereafter, a radioactive emission detector, such as or a gamma camera, SPECT, or PET, is employed for locating the position of the active area.

In addition to detecting tumors and pathologies, radiopharmacueticals such as ACU TECT from Nycomed Amersham, may be used in the detection of newly formed thrombosis in veins or clots in arteries of the heart or brain, in an emergency or operating room. Yet other applications include radioimaging of myocardial infarct using agents such as radioactive anti-myosin antibodies, radioimaging specific cell types using radioactively tagged molecules (also known as molecular imaging), etc.

The distribution image of the radiopharmaceutical in and around a tumor, or another body structure, is obtained by recording the radioactive emission of the radiopharmaceutical with an external or intracorporeal radiation detector placed at different locations outside or inside the patient. The usual preferred emission for such applications is that of gamma rays, which emission is in the energy range of approximately 20-511 KeV. When the probe is placed in contact with the tissue, beta radiation and positrons may also be detected.

The first attempts at radionuclide “imaging” were in the late 1940's. An array of radiation detectors was positioned mechanically on a matrix of measuring points around the head of a patient. Alternatively, a single detector was positioned mechanically for separate measurements at each point on the matrix.

A significant advance occurred in the early 1950's with the introduction of the rectilinear scanner by Ben Cassen. With this instrument, the detector was scanned mechanically in a predetermined pattern over the area of interest.

The first gamma camera capable of recording all points of the image at one time was described by Hal Anger in 1953. Anger used a detector comprised of a NaI(T1) screen and a sheet of X-ray film. In the late 1950's, Anger replaced the film screen with a photomultiplier tube assembly. The Anger camera is described in Hal 0. Anger, “Radioisotope camera in Hine G J”, Instrumentation in Nuclear Medicine, New York, Academic Press 1967, chapter 19. U.S. Pat. No. 2,776,377 to Anger, issued in 1957, also describes such a radiation detector assembly.

U.S. Pat. No. 4,959,547 to Carroll et al. describes a probe used to map or provide imaging of radiation within a patient. The probe comprises a radiation detector and an adjustment mechanism for adjusting the solid angle through which radiation may pass to the detector, the solid angle being continuously variable. The probe is constructed so that the only radiation reaching the detector is that which is within the solid angle. By adjusting the solid angle from a maximum to a minimum while moving the probe adjacent the source of radiation and sensing the detected radiation, one is able to locate the probe at the source of radiation. The probe can be used to determine the location of the radioactivity and to provide a point-by-point image of the radiation source or data for mapping the same.

U.S. Pat. No. 5,246,005 to Carroll et al. describes a radiation detector or probe, which uses statistically valid signals to detect radiation signals from tissue. The output of a radiation detector is a series of pulses, which are counted for a predetermined amount of time. At least two count ranges are defined by circuitry in the apparatus and the count range which includes the input count is determined. For each count range, an audible signal is produced which is audibly distrainable from the audible signal produced for every other count range. The mean values of each count range are chosen to be statistically different, e.g., 1, 2, or 3 standard deviations, from the mean of adjacent lower or higher count ranges. The parameters of the audible signal, such as frequency, voice, repetition rate, and (or) intensity are changed for each count range to provide a signal, which is discriminable from the signals of any other count range.

U.S. Pat. No. 5,475,219 to Olson describes a system for detecting photon emissions wherein a detector serves to derive electrical parameter signals having amplitudes corresponding with the detected energy of the photon emissions and other signal generating events. Two comparator networks employed within an energy window, which define a function to develop an output, L, when an event-based signal amplitude is equal to or above a threshold value, and to develop an output, H, when such signal amplitude additionally extends above an upper limit. Improved reliability and accuracy is achieved with a discriminator circuit which, in response to these outputs L and H, derives an event output upon the occurrence of an output L in the absence of an output H. This discriminator circuit is an asynchronous, sequential, fundamental mode discriminator circuit with three stable states.

U.S. Pat. Nos. 5,694,933 and 6,135,955 to Madden et al. describe a system and method for diagnostic testing of a structure within a patient's body that has been provided with a radioactive imaging agent, e.g., a radiotracer, to cause the structure to produce gamma rays, associated characteristic x rays, and a continuum of Compton-scattered photons. The system includes a radiation-receiving device, e.g., a hand-held probe or camera, an associated signal processor, and an analyzer. The radiation receiving device is arranged to be located adjacent the body and the structure for receiving gamma rays and characteristic X-rays emitted from the structure and for providing a processed electrical signal representative thereof. The processed electrical signal includes a first portion representing the characteristic X-rays received and a second portion representing the gamma rays received. The signal processor removes the signal corresponding to the Compton-scattered photons from the electrical signal in the region of the full-energy gamma ray and the characteristic X-ray. The analyzer is arranged to selectively use the X-ray portion of the processed signal to provide near-field information about the structure, to selectively use both the X-ray and the gamma-ray portions of the processed signal to provide near-field and far-field information about the structure, and to selectively use the gamma-ray portion of the processed signal to provide extended field information about the structure.

U.S. Pat. No. 5,732,704 to Thurston et al. describes a method for identifying a sentinel lymph node located within a grouping of regional nodes at a lymph drainage basin associated with neoplastic tissue wherein a radiopharmaceutical is injected at the situs of the neoplastic tissue. This radiopharmaceutical migrates along a lymph duct towards the drainage basin containing the sentinel node. A hand-held probe with a forwardly disposed radiation detector crystal is maneuvered along the duct while the clinician observes a graphical readout of count rate amplitudes to determine when the probe is aligned with the duct. The region containing the sentinel node is identified when the count rate at the probe substantially increases. Following surgical incision, the probe is maneuvered utilizing a sound output in connection with actuation of the probe to establish increasing count rate thresholds followed by incremental movements until the threshold is not reached and no sound cue is given to the surgeon. At this point of the maneuvering of the probe, the probe detector will be in adjacency with the sentinel node, which then may be removed.

U.S. Pat. No. 5,857,463 to Thurston et al. describes further apparatus for tracking a radiopharmaceutical present within the lymph duct and for locating the sentinel node within which the radiopharmaceutical has concentrated. A smaller, straight, hand-held probe is employed carrying two hand actuable switches. For tracking procedures, the probe is moved in an undulatory manner, wherein the location of the radiopharmaceutical-containing duct is determined by observing a graphics readout. When the region of the sentinel node is approached, a switch on the probe device is actuated by the surgeon to carry out a sequence of squelching operations until a small node locating region is defined.

U.S. Pat. No. 5,916,167 to Kramer et al. and U.S. Pat. No. 5,987,350 to Thurston describe surgical probes wherein a heat-sterilizable and reusable detector component is combined with a disposable handle and cable assembly. The reusable detector component incorporates a detector crystal and associated mountings along with preamplifier components.

U.S. Pat. No. 5,928,150 to Call describes a system for detecting emissions from a radiopharmaceutical injected within a lymph duct wherein a hand-held probe is utilized. When employed to locate sentinel lymph nodes, supplementary features are provided including a function for treating validated photon event pulses to determine count rate level signals. The system includes a function for count-rate based ranging as well as an adjustable thresholding feature. A post-threshold amplification circuit develops full-scale aural and visual outputs. U.S. Pat. Nos. 5,932,879 and 6,076,009 to Raylman et al. describe an intraoperative system for preferentially detecting beta radiation over gamma radiation emitted from a radiopharmaceutical. The system has ion-implanted silicon charged-particle detectors for generating signals in response to received beta particles. A preamplifier is located in proximity to the detector filters and amplifies the signal. The probe is coupled to a processing unit for amplifying and filtering the signal.

U.S. Pat. No. 6,144,876 to Bouton describes a system for detecting and locating sources of radiation, with particular applicability to interoperative lymphatic mapping (ILM) procedures. The scanning probe employed with the system performs with both an audible as well as a visual perceptive output. A desirable stability is achieved in the readouts from the system through a signal processing approach which establishes a floating or dynamic window analysis of validated photon event counts. This floating window is defined between an upper edge and a lower edge. The values of these window edges vary during the analysis in response to compiled count sum values. In general, the upper and lower edges are spaced apart a value corresponding with about four standard deviations.

To compute these count sums, counts are collected over successive short scan intervals of 50 milliseconds and the count segments resulting therefrom are located in a succession of bins within a circular buffer memory. The count sum is generated as the sum of the memory segment count values of a certain number of the bins or segments of memory. Alteration of the floating window occurs when the count sum either exceeds its upper edge or falls below its lower edge. A reported mean, computed with respect to the window edge that is crossed, is developed for each scan interval which, in turn, is utilized to derive a mean count rate signal. The resulting perceptive output exhibits a desirable stability, particularly under conditions wherein the probe detector is in a direct confrontational geometry with a radiation source.

U.S. Pat. No. 5,846,513 teaches a system for detecting and destroying living tumor tissue within the body of a living being. The system is arranged to be used with a tumor localizing radiopharmaceutical. The system includes a percutaneously insertable radiation detecting probe, an associated analyzer, and a percutaneously insertable tumor removing instrument, e.g., a resectoscope. The radiation detecting probe includes a needle unit having a radiation sensor component therein and a handle to which the needle unit is releasably mounted. The needle is arranged to be inserted through a small percutaneous portal into the patient's body and is movable to various positions within the suspected tumor to detect the presence of radiation indicative of cancerous tissue. The probe can then be removed and the tumor removing instrument inserted through the portal to destroy and (or) remove the cancerous tissue. The instrument not only destroys the tagged tissue, but also removes it from the body of the being so that it can be assayed for radiation to confirm that the removed tissue is cancerous and not healthy tissue. A collimator may be used with the probe to establish the probe's field of view.

The main limitation of the system is that once the body is penetrated, scanning capabilities are limited to a translational movement along the line of penetration.

An effective collimator for gamma radiation must be several mm in thickness and therefore an effective collimator for high-energy gamma radiation cannot be engaged with a fine surgical instrument such as a surgical needle. On the other hand, beta radiation is absorbed mainly due to its chemical reactivity after passage of about 0.2-3 mm through biological tissue. Thus, the system described in U.S. Pat. No. 5,846,513 cannot efficiently employ high-energy gamma detection because directionality will to a great extent be lost and it also cannot efficiently employ beta radiation because too high proximity to the radioactive source is required, whereas body tissue limits the degree of maneuvering the instrument.

The manipulation of soft tissue organs requires visualization (imaging) techniques such as computerized tomography (CT), fluoroscopy (X-ray fluoroscopy), magnetic resonance imaging (MRI), optical endoscopy, mammography or ultrasound which distinguish the borders and shapes of soft tissue organs or masses. Over the years, medical imaging has become a vital part in the early detection, diagnosis and treatment of cancer and other diseases. In some cases medical imaging is the first step in preventing the spread of cancer through early detection and in many cases medical imaging makes it possible to cure or eliminate the cancer altogether via subsequent treatment.

An evaluation of the presence or absence of tumor metastasis or invasion has been a major determinant for the achievement of an effective treatment for cancer patients. Studies have determined that about 30% of patients with essentially newly diagnosed tumor will exhibit clinically detectable metastasis. Of the remaining 70% of such patients who are deemed “clinically free” of metastasis, about one-half are curable by local tumor therapy alone. However, some of these metastasis or even early stage primary tumors do not show with the imaging tools described above. Moreover often enough the most important part of a tumor to be removed for biopsy or surgically removed is the active, i.e., growing part, whereas using only conventional imaging cannot distinguish this specific part of a tumor from other parts thereof and (or) adjacent non affected tissue.

A common practice in order to locate this active part is to mark it with radioactivity tagged materials generally known as radiopharmaceuticals, which are administered orally or intravenously and which tend to concentrate in such areas, as the uptake of such radiopharmaceuticals in the active part of a tumor is higher and more rapid than in the neighboring tumor tissue. Thereafter, a radioactive emission detector is employed for locating the position of the active area.

Medical imaging is often used to build computer models, which allow doctors to, for example, guide exact radiation in the treatment of cancer, and to design minimally-invasive or open surgical procedures. Moreover, imaging modalities are also used to guide surgeons to the target area inside the patient's body, in the operation room during the surgical procedure. Such procedures may include, for example, biopsies, inserting a localized radiation source for direct treatment of a cancerous lesion, known as brachytherapy (so as to prevent radiation damage to tissues near the lesion), injecting a chemotherapy agent into the cancerous site or removing a cancerous or other lesions.

The aim of all such procedures is to pinpoint the target area as precisely as possible in order to get the most precise biopsy results, preferably from the most active part of a tumor, or to remove such a tumor in its entirety on the one hand with minimal damage to the surrounding, non affected tissues, on the other hand. Therefore, there is a persistent need to improve available imaging techniques.

### SUMMARY OF THE INVENTION

According to one aspect of the present invention, there is thus provided an intracorporeal-imaging head, comprising:

a housing, which comprises:

a first optical imaging system, mounted on the housing, adapted to optically image a portion of a tissue; and

at least one radioactive-emission probe, mounted on the housing, adapted to image radioactive-emission from the portion.

According to an additional aspect of the present invention, the intracorporeal-imaging head comprises a position-tracking device, mounted on the housing, in a fixed positional relation with the radioactive-emission probe, for providing positional information for the radioactive-emission probe.

According to an additional aspect of the present invention, the position-tracking device has six degrees of freedom.

According to an additional aspect of the present invention, the intracorporeal-imaging head is adapted for obtaining high-resolution, radioactive-emission imaging by collimation-deconvolution algorithms.

According to an additional aspect of the present invention, the first optical imaging system includes:

a lighting system, adapted to shine light on intracorporeal objects;

an lens, for focusing images of the intracorporeal objects; and

a light detecting array, for detecting the images of the intracorporeal objects.

According to an additional aspect of the present invention, the first optical imaging system is adapted for wide-angle imaging, at an angle greater than substantially 49 degrees.

According to an alternative or an additional aspect of the present invention, the first optical imaging system is adapted for normal imaging, at an angle of substantially 49 degrees.

According to an alternative or an additional aspect of the present invention, the first optical imaging system is adapted for zoom imaging, at an angle smaller than substantially 49 degrees.

According to an additional aspect of the present invention, the lighting system is a laser lighting system.

According to an additional aspect of the present invention, the lighting system is an infrared lighting system.

According to an alternative or an additional aspect of the present invention, the lighting system is substantially a white-light lighting system.

According to an additional aspect of the present invention, the intracorporeal-imaging head comprises a second optical imaging system, adapted for zooming in on suspected pathologies, identified by the first optical imaging system.

According to an additional aspect of the present invention, the second optical imaging system is a video camera.

According to an alternative or an additional aspect of the present invention, the second optical imaging system is a still camera.

According to an additional aspect of the present invention, the radioactive-emission probe is a single-pixel probe.

According to an additional aspect of the present invention, the radioactive-emission probe is a single-pixel, collimated probe.

According to an additional aspect of the present invention, the single-pixel, collimated probe has a tube collimator.

According to an additional aspect of the present invention, the single-pixel, collimated probe has a wide-angle collimator.

According to an alternative aspect of the present invention, the radioactive-emission probe is a multi-pixel probe.

According to an additional aspect of the present invention, the radioactive-emission probe is a multi-pixel, collimated probe.

According to an additional aspect of the present invention, the multi-pixel, collimated probe has tube collimators.

According to an alternative aspect of the present invention, the multi-pixel, collimated probe has wide-angle collimators.

According to an alternative aspect of the present invention, the housing is tubular, and the radioactive-emission probe is a multi-pixel probe, with detector pixels arranged radially about a center, each pixel having a collimator.

According to an additional aspect of the present invention, the collimators are rectangular.

According to an alternative aspect of the present invention, the collimators fan out, in a manner similar to flower petals.

According to an additional aspect of the present invention, the at least one radioactive-emission probe comprises a plurality of radioactive-emission probes.

According to an additional aspect of the present invention, the intracorporeal-imaging head comprises at least one ultrasound-imaging device.

According to an additional aspect of the present invention, the intracorporeal-imaging head comprises an MRI imaging device.

According to an additional aspect of the present invention, the intracorporeal-imaging head is adapted for rotation.

According to an additional aspect of the present invention, the intracorporeal-imaging head is adapted to be mounted on an endoscope for insertion through a trucar valve.

According to an additional aspect of the present invention, the intracorporeal-imaging head is adapted to be mounted on an endoscope for insertion through a body lumen.

According to an additional aspect of the present invention, the intracorporeal-imaging head is adapted to be mounted on a resectoscope for insertion through a urinary tract.

According to an additional aspect of the present invention, the intracorporeal-imaging head is adapted to be mounted on a colonoscope.

According to an additional aspect of the present invention, the intracorporeal-imaging head comprises a surgical instrument.

According to an additional aspect of the present invention, the first optical imaging system is a video camera.

According to an additional aspect of the present invention, the first optical imaging system is a still camera.

According to another aspect of the present invention, there is thus provided a method of intracorporeal imaging, comprising:

providing an imager;

performing a first optical imaging of an intracorporeal portion of a tissue, by the imager; and

performing a radioactive-emission imaging of the portion, by the imager.

According to still another aspect of the present invention, there is thus provided an intracorporeal-detecting head, comprising:

a housing, which comprises:

a first optical detecting system, mounted on the housing, adapted to optically view a portion of a tissue; and

at least one radioactive-emission probe, mounted on the housing, adapted to detect radioactive-emission from the portion.

According to yet another aspect of the present invention, there is thus provided a method of intracorporeal detecting, comprising:

providing an detector;

performing a first optical detecting of an intracorporeal portion of a tissue, by the detector; and

performing a radioactive-emission detecting of the portion, by the detector.

The present invention successfully addresses the shortcomings of the presently known configurations by providing an intracorporeal-imaging head, which combines at least optical and radioactive-emission imaging, possibly also with high-resolution position tracking. The radioactive-emission-imaging probe has a wide-aperture, or coarse collimator, for high count-rate efficiency; nevertheless, the high-resolution position tracking ensures high resolution of the radioactive-emission image. Specifically, wide-aperture collimation-deconvolution algorithms are provided, for obtaining a high-efficiency, high resolution image of a radioactive-emission source, by scanning the radioactive-emission source with a probe of a wide-aperture collimator, and at the same time, monitoring the position of the radioactive-emission probe, at very fine time intervals, to obtain the equivalence of fine-aperture collimation. The blurring effect of the wide aperture is then corrected mathematically. The intracorporeal-imaging head may further include ultrasound and MRI imagers, as well as a surgical instrument, such as a biopsy needle, a knife, a cryosurgery device, a resection wire, a laser ablation device, an ultrasound ablation device, other devices for localized radiation ablations, devices for implanting brachytherapy seeds, and other minimally invasive devices. According to another embodiment, an intracorporeal-detecting head is provided, which combines at least optical and radioactive-emission detectors, for a “Yes or No” type detection, by the at least two modalities.

Implementation of the methods and systems of the present invention involves performing or completing selected tasks or steps manually, automatically, or a combination thereof. Moreover, according to actual instrumentation and equipment of preferred embodiments of the methods and systems of the present invention, several selected steps could be implemented by hardware or by software on any operating system of any firmware or a combination thereof. For example, as hardware, selected steps of the invention could be implemented as a chip a circuit. As software, selected steps of the invention could be implemented as a plurality of software instructions being executed by a computer using any suitable algorithms. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1C schematically illustrate the effects of a collimator's geometry on the counting efficiency of a radioactive-emission imaging system, as known;

FIG. 2 schematically illustrates the components of a coarse-collimator, high-resolution radioactive-emission imaging system, in accordance with the present invention;

FIG. 3 schematically illustrates the manner of operation of the coarse-collimator, high-resolution radioactive-emission imaging system 20 of FIG. 2, in accordance with the present invention;

FIGS. 4A-4C schematically illustrate data acquisition in a one-dimensional space, in accordance with the present invention;

FIGS. 5A-5C schematically illustrate data acquisition in a two-dimensional space, in accordance with the present invention;

FIGS. 6A-6D schematically illustrate data acquisition in a three-dimensional space, in accordance with the present invention;

FIGS. 7A-7K schematically illustrate imaging heads, for imaging at least by optical and radioactive-emission modalities, operable with endoscopes, colonoscopes and resectoscopes, in accordance with the present invention;

FIG. 8 schematically illustrates an endoscope for minimally invasive surgery, adapted for insertion via a trucar valve, for imaging at least by optical and radioactive-emission modalities, in accordance with the present invention;

FIG. 9 schematically illustrates an endoscope, adapted for insertion in a body lumen, for imaging at least by optical and radioactive-emission modalities, in accordance with the present invention;

FIG. 10 schematically illustrates a resectoscope, adapted for insertion via the urinary track, for imaging at least by optical and radioactive-emission modalities, in accordance with the present invention; and

FIGS. 11A and 11B schematically illustrate a colonoscope, for imaging at least by optical and radioactive-emission modalities, in accordance with the present invention.

### DESCRIPTION OF THE PREFERRED EMBODIMENTS

The present invention is of an intracorporeal-imaging head, which combines at least optical and radioactive-emission imaging, possibly also with high-resolution position tracking. The radioactive-emission-imaging probe has a wide-aperture collimator, for high count-rate efficiency; nevertheless, the high-resolution position tracking ensures high resolution of the radioactive-emission image. Specifically, wide-aperture collimation-deconvolution algorithms are provided, for obtaining a high-efficiency, high resolution image of a radioactive-emission source, by scanning the radioactive-emission source with a probe of a wide-aperture collimator, and at the same time, monitoring the position of the radioactive-emission probe, at very fine time intervals, to obtain the equivalence of fine-aperture collimation. The blurring effect of the wide aperture is then corrected mathematically. The intracorporeal-imaging head may further include ultrasound and MRI imagers, as well as a surgical instrument, such as a biopsy needle, a knife, a cryosurgery device, a resection wire, a laser ablation device, an ultrasound ablation device, other devices for localized radiation ablations, devices for implanting brachytherapy seeds, and other minimally invasive devices. According to another embodiment, an intracorporeal-detecting head is provided, which combines at least optical and radioactive-emission detectors, for a “Yes or No” type detection, by the at least two modalities.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

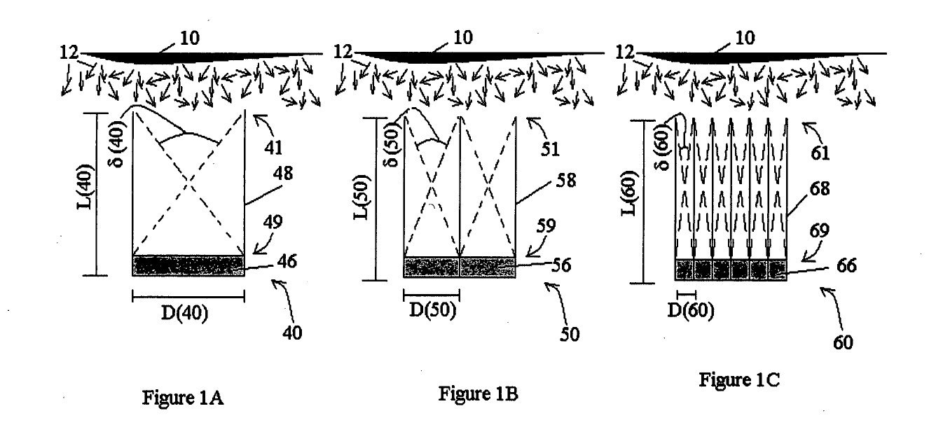

Referring now to the drawings, FIGS. 1A-6D schematically illustrate the principle of radioactive-emission-imaging, such as gamma imaging, with wide-aperture, or coarse collimation and high-resolution position tracking, and image reconstruction, which includes deconvolution, for resolution enhancement. The overall algorithms are herethereto referred to as collimation-deconvolution algorithms.

In general, optimization of a gamma-imaging probe requires optimizing both count rate efficiency and image resolution. The first in necessary in order to detect low-radiation sources, while the second provides information on the size and extent of the source. Yet these tend to work against each other. Count-rate efficiency is increased with increasing collimator's viewing angle, while image resolution is increased with decreasing collimator's viewing angle, as seen in FIGS. 1A-1C, illustrating three collimation geometries, adapted to image a radioactive-emission source 10, emitting radiation 12.

FIG. 1A illustrates a first geometry of a first probe 40, having a single-pixel detector 46 and a collimator 48, adapted to image radioactive-emission source 10, emitting radiation 12. Defining N(40) as the number of pixels, N(40)=1. The collimator's length is L(40) and the collimator's width is D(40), wherein the ratio of L/D determines the viewing angle. Given, for example, that L(40)/D(40)=2, a viewing angle δ(40) is substantially 70 degrees; probe 40 is sensitive to incident photons within the confinement of about an 70-degree arc, but the single pixel provides no resolution to speak of.

FIG. 1B illustrates a second geometry of a second probe 50, having a two-pixel detector 56, each associated with a collimator 58, of a length L(50) and a width D(50). Defining N(50) as the number of pixels, N(50)=2. Probe 50 has the same overall dimensions as probe 40, but L(50)/D(50)=4. A viewing angle δ(50) is substantially 50 degrees; probe 50 is sensitive to incident photons within the confinement of about an 50-degree arc, while the two pixels provide a limited resolution.

FIG. 1C illustrates a third geometry of a third probe 60, having a six-pixel detector 66, each associated with a collimator 68, of a length L(60) and a width D(60). Defining N(60) as the number of pixels, N(60)=2. Probe 60 has the same overall dimensions as probe 40, thus, L(60)/D(60)=12. A viewing angle δ(60) is substantially 9 degrees; each pixel of probe 60 is sensitive to incident photons within the confinement of about an 9-degree arc, while providing a resolution of six pixels.

In other words, while a wide-aperture, single-pixel probe provides high efficiency, it does not lend itself to the generation of a two-dimensional image, and the wide aperture blurs the information regarding the direction from which the radiation comes. Yet when the resolution is increased, the efficiency is decreased.

However, in accordance with the teachings of the present invention, if the movement of probe 40 in space is recorded with a high resolution, so that the position and angular orientation of probe 40 are accurately tracked, at very short time intervals, for example, of about 100 or 200 ms, high resolution, one-, two-, and three-dimensional images of counting rate as a function of position can be obtained, by data processing.

More specifically, knowing the position and orientation of probe 40 at each time interval and the radioactive-emission count rate at that position and orientation, two- and three-dimensional images of the radioactive-emission density can be constructed. Additionally, while it is known that the information is blurred, or convolved, by the wide-aperture collimator, a deconvolution process may be used to obtain dependable results. Moreover, the convolving function for a wide aperture depends only on the geometry of the collimator and may be expressed as a set of linear equations that can be readily solved, by collimation-deconvolution algorithms, described for example, in commonly owned U.S. patent application Ser. No. 10/343,792, Publication No. 20040015075, whose disclosure is incorporated herein by reference.

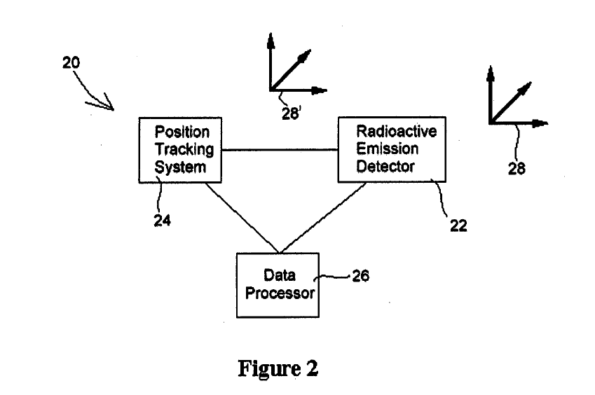

FIG. 2 schematically illustrates the components of a coarse-collimator, high-resolution radioactive-emission imaging system 20, in accordance with the present invention.

System 20 includes three major components, in communication with each other: a radioactive-emission probe 22, a position tracking system 24, and a data processor 26. Radioactive-emission probe 22 is moving in a coordinate system 28, while position tracking system 24 is moving in a coordinate system 28′, which is recorded, and which is in a fixed and known relations with coordinate system 28. Preferably, radioactive-emission probe 22 is a single-pixel probe, or a coarse-grid probe, so as to provide no resolution information. Yet high resolution may be obtained by integrating position tracking with radioactive emission count rate, at very fine time intervals.

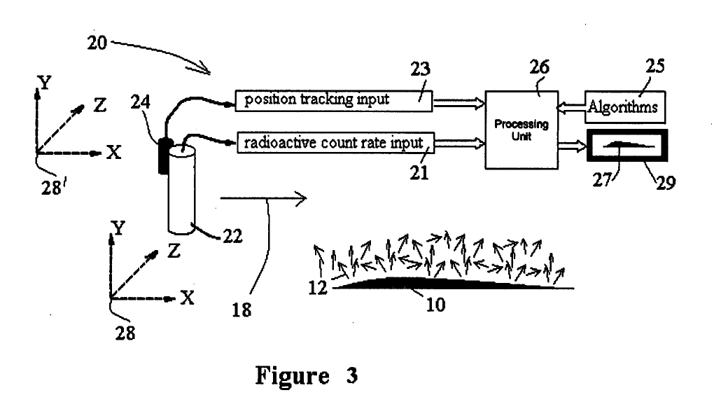

FIG. 3 schematically illustrates the manner of operation of the coarse-collimator, high-resolution radioactive-emission imaging system 20 of FIG. 2, moving about radioactive-emission source 10, emitting radiation 12, as indicated by an arrow 18, in accordance with the present invention.

Data processor 26 receives time-dependent position-tracking input 23 from position tracking system 24 moving in coordinate system 28′, and single-pixel, or coarse-pixel radioactive-emission count-rate input 21, for each time interval, from radioactive-emission probe 22, moving in coordinate system 28. Using collimation-deconvolution algorithms 25, data processor 26 generates a radioactive-emission image 27, for example, on a display screen 29.

Thus, the effective number of pixels of image 27 is based on the number of time intervals, at which simultaneous radioactive count-rate and position input were taken. While in ordinary cameras, image resolution depends on the number of pixels of the camera, in accordance with the present invention, image resolution depends on the accuracy of the position tracking system and on the fineness of the time intervals, at which simultaneous position-tracking input 23 and radioactive count-rate input 21 are taken.

In consequence, a high-efficiency, wide-aperture, coarse, or wide-bore collimator probe, having a position tracking system, operating at very short time intervals, and connected to a data processor for wide-aperture collimation-deconvolution algorithms, can be used for the construction of a high-resolution image of radiation density in one-, two-, or three-dimensions.

An example of a suitable position tracking system 24 is miniBird™, which is a magnetic tracking and location system commercially available from Ascension Technology Corporation, P.O. Box 527, Burlington, Vt. 05402 USA (http://www.ascension-tech.com/graphic.htm). The miniBird™ measures the real-time position and orientation (six degrees of freedom) of one or more miniaturized sensors, so as to accurately track the spatial location of probes, instruments, and other devices. The dimensions of miniBird™ 158 are 18 mm×8 mm×8 mm for Model 800 and 10 mm×5 mm×5 mm the Model 500. Alternatively, optical tracking systems, of Northern Digital Inc., Ontario, Canada NDI-POLARIS which provides passive or active systems, magnetic tracking systems of NDI-AURORA, infrared tracking systems of E-PEN system, http://www.e-pen.com, or ultrasonic tracking systems of E-PEN system may be used.

Preferably, radioactive-emission probe 22 is constructed as single-pixel probe 40 (FIG. 1A), with detector 46 being formed of room temperature CdZnTe, obtained, for example, from eV Products, a division of II-VI Corporation, Saxonburg Pa., 16056. Alternatively, another solid-state detector such as CdTe, HgI, Si, Ge, or the like, or a scintillation detector, such as NaI(T1), LSO, GSO, CsI, CaF, or the like, or another detector as known, may be used.

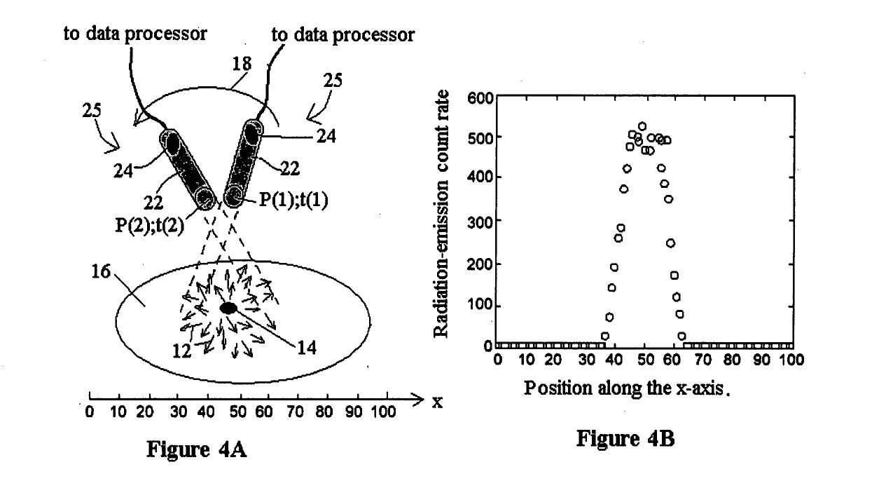

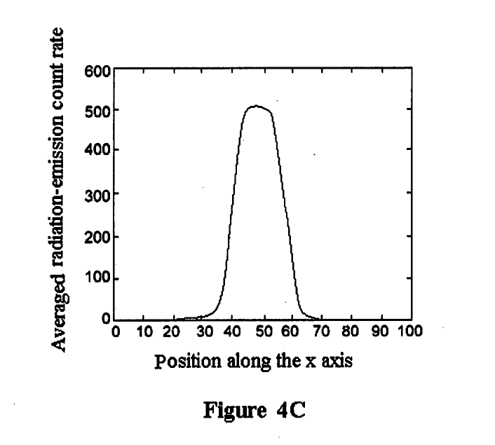

FIGS. 4A-4C schematically illustrate data acquisition in a one-dimensional space, in accordance with the present invention.

As seen in FIG. 4A, an imager 25, formed of radioactive-emission probe 22 coupled with position tracking system 24, is used to image a point source 14 emitting radiation 12 within a control area 16. Imager 25 may move along one or several paths, for example, path 18, for imaging radioactive emission along an x-axis. At a time t(1), imager 25 is located at P(1). At a time t(2), imager 25 is located at P(2). Time-dependent position-tracking input 23 (FIG. 3) and radioactive count-rate input 21 are forwarded to data processor 26, at intervals Δt.

As seen in FIG. 4B, discrete data may be plotted as count rate as a function of position, along the x-axis. A peak count rate around x=50 mm is indicative of a point source at that location. Yet, because of the discrete nature of the data, it may be desirable to smooth it out with an averaging algorithm.

Averaging may be achieved, for example, by averaging each new count, N(x+Δx), at a position x+Δx with the previous count N(x) at a position x, while taking into account the physical parameters of imager 25, which include the physical parameters of radioactive emission probe 22, the physical parameters of position tracking system 24, and the physical relation between radioactive emission probe 22 and position tracking system 24.

As seen in FIG. 4C, a smooth curve of averaged count rate as a function of position along the x-axis may thus be achieved. The smooth curve is more conducive to analysis, since it may be described by a mathematical expression and may be used in constructing a two- or a three-dimensional image.

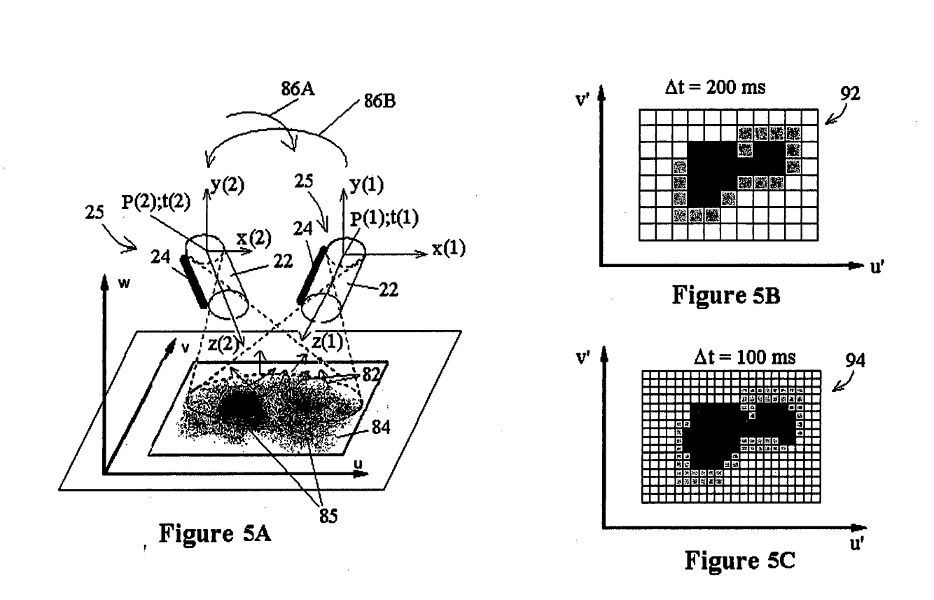

FIGS. 5A-5C schematically illustrate data acquisition in a two-dimensional space, in accordance with the present invention.

As seen in FIG. 5A, imager 25, formed of radioactive-emission probe 22 coupled with position tracking system 24, is used to image a control area 84, which includes a radiation source 85, emitting radiation 82, in a system of coordinates u;v. Imager 25 may move along several paths, for example, 86A and 86B. At a time t(1), imager 25 is located at P(1) having coordinates x(1);y(1). At a time t(2), imager 25 is located at P(2) having coordinates x(2);y(2). Time-dependent position-tracking input 23 (FIG. 3) and radioactive count-rate input 21 are forwarded to data processor 26, at intervals Δt.

FIG. 5B illustrates a two-dimensional image 92, that was formed by data processor 26, in system of coordinates u′;v′, assuming for example, time intervals Δt of 200 ms.

FIG. 5C illustrates a two dimensional image 94, of higher resolution than image 92, formed by data processor 26, in system of coordinates u′;v′, using the same imager 25, and time intervals Δt of 100 ms. As FIGS. 5B and 5C illustrate, when within the bounds of position tracking resolution of position tracking system 24, the resolution of count rate as a function of position is controlled by the fineness of the time intervals, at which simultaneous position-tracking input 23 (FIG. 3) and radioactive count-rate input 21 are taken.

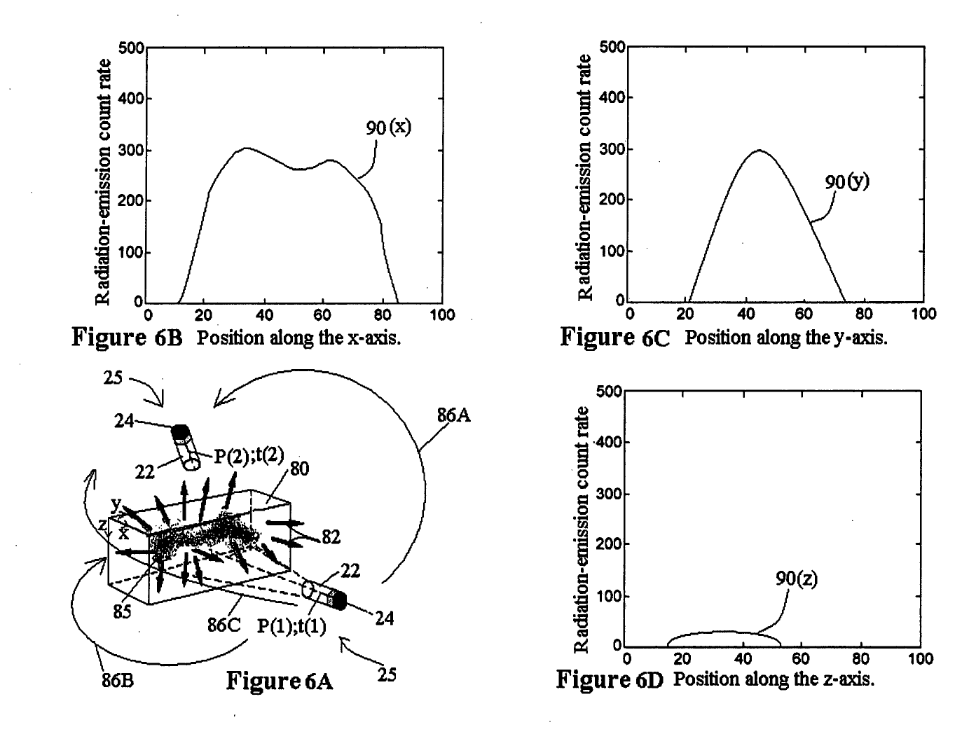

FIGS. 6A-6D schematically illustrate data acquisition in a three-dimensional space, in accordance with the present invention.

As seen in FIG. 6A, imager 25 moves around control volume 80, which includes radiation source 85, emitting radiation 82, in a system of coordinates x;y;z. Imager 25 may move along several paths, for example, 86A, 86B, and 86C, and its motion is monitored in two, three and up to six dimensions—the linear x-, y- and z-axes and the rotational angles ρ, θ and φ, about them, respectively.

FIGS. 6B-6D illustrate images 90(x), 90(y), and 90(z), of averaged count rate as a function of position, in x-, y-, and z-axes.

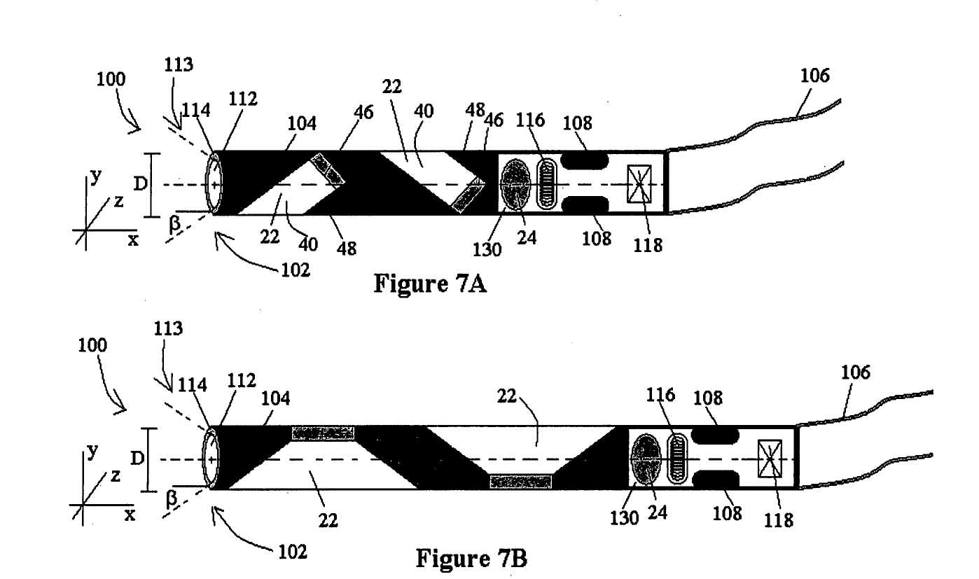

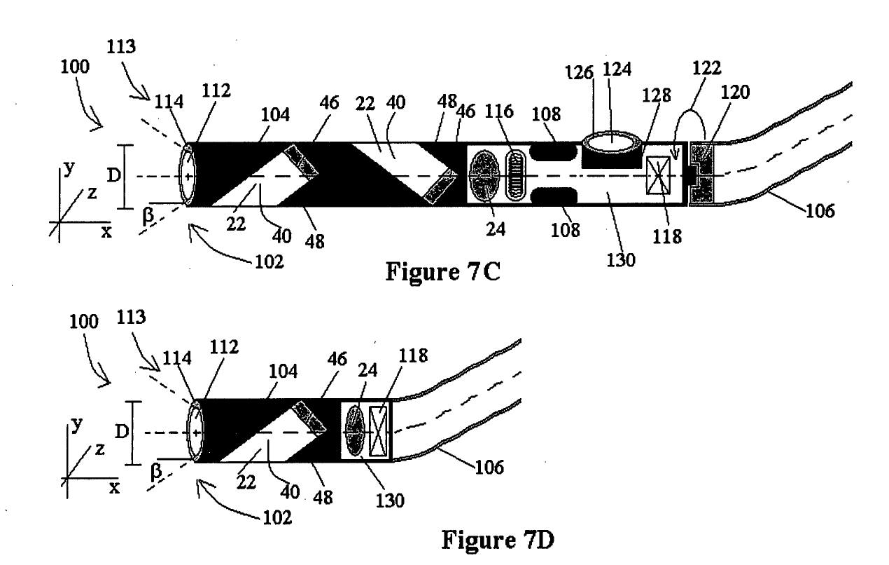

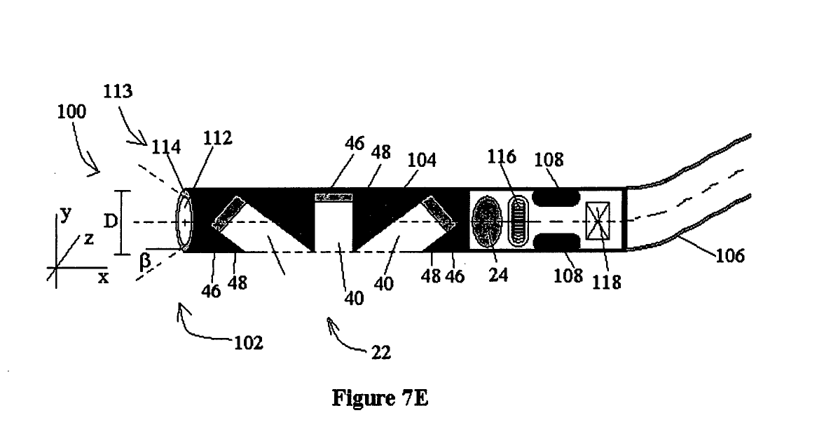

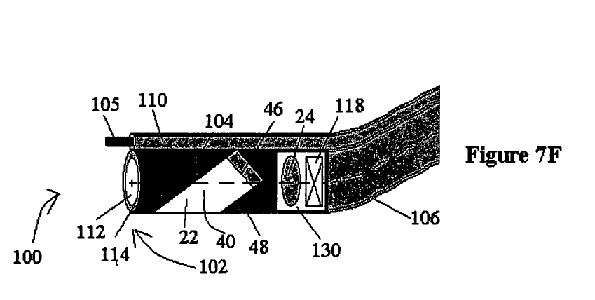

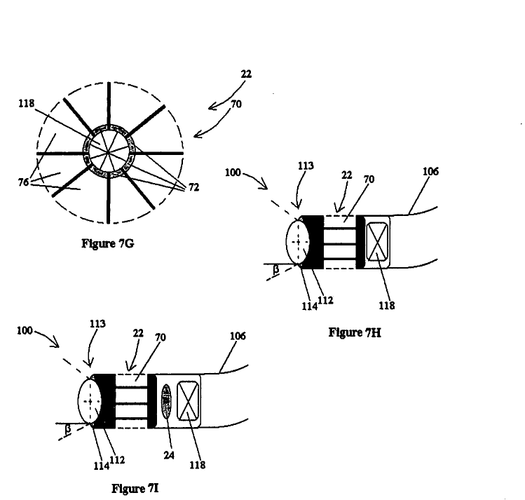

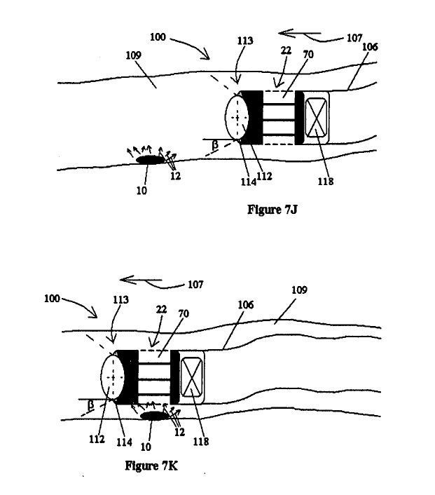

Referring further to the drawings, FIGS. 7A-7K schematically illustrate an intracorporeal-imaging head 100, which combines at least optical and radioactive-emission imaging, in accordance with the present invention.

As seen in FIG. 7A, intracorporeal-imaging head 100, having a distal end 102, with respect to an operator, defines an x;y;z coordinate system. It is preferably formed as a tube 130, for example, of stainless steel, titanium or another biocompatible material, metal or alloy. An overall diameter D of tube 130 may be for example, between 5 and 25 cm, depending on the application.

Intracorporeal-imaging head 100 includes a camera 113, preferably a video camera, comprising a lens 112 and preferably also a lighting 114. Alternatively, camera 113 may be a still camera. Preferably, camera 113 is located at distal end 102. Lens 112 may be a normal lens, a wide-angle lens, a telescopic lens, or a zoom lens of a variable viewing angle β. Lighting 114 may be a white light, an infrared light, or both, and may be one or several light diodes, laser light diodes or one or several optical fiber ends, for transmitting light.

Additionally, intracorporeal-imaging head 100 includes at least one, and preferably several radioactive-emission probes 22, preferably, formed as single-pixel radioactive-emission probe 40, of FIG. 1A, each having radiation detector 46 and collimator 48, formed, for example, as a tube. Preferably, several radioactive-emission probes 22 are each pointing at a different direction. In a preferred embodiment, radioactive-emission probes 22 are placed in a solid cylinder of lead 104, into which holes have been drilled to house radioactive-emission probes 22 and to provide channels (not shown) for the necessary electronics.

Additionally, intracorporeal-imaging head 100 may include a position-tracking system 24, and possibly also, one or several ultrasound transducers 108, an MRI probe 116 and related electronics 118.

Intracorporeal-imaging head 100 may be attached to catheter 106, formed for example, as a flexible tubing.

Preferably, position-tracking system 24 is miniBird™, the magnetic tracking and location system of Ascension Technology Corporation, of Burlington, Vt. Alternatively, other systems, as known, may be used.

Radioactive-emission probe 22 may be single-pixel probe 40, of FIG. 1A. Alternatively, another single-pixel probe or a multi-pixel probe may be used. Detector 46 may be formed of room temperature CdZnTe, obtained, for example, from eV Products, a division of II-VI Corporation, Saxonburg Pa., 16056. Alternatively, other detectors may be used.

FIG. 7B illustrates a somewhat different arrangement, wherein radioactive-emission probes 22 have wide-angle collimators.

It will be appreciated that other geometries are also possible. For example, radioactive-emission probe 22 may be constructed as a grid, for example, a square grid, of several pixels. Additionally or alternatively, radioactive-emission probe 22 may be constructed with a narrow-angle collimator.

FIG. 7C illustrates a still different arrangement, wherein intracorporeal-imaging head 100 is connected to catheter 106 via a motor 120, and may be rotated around the x-axis, as shown by arrow 122. The purpose of the rotation is to enable radioactive-emission probes 22, which are fixed within intracorporeal-imaging head 100, to scan in every direction.

Additionally, a second camera 128 having a preferably zoom lens 124 and a lighting 126 may be provided. Camera 128 may be a video or a still camera. Additionally, lighting 126 may be a white light, an infrared light, or a combination of both. Preferably, if camera 113 identifies a suspected pathology, intracorporeal-imaging head 100 may be rotated so as to enable both radioactive-emission probes 22 and second camera 128 to image the pathology more closely and more attentively.

Alternatively, as seen in FIG. 7D, intracorporeal-imaging head 100 may include only camera 113, one radioactive-emission probe 22, related electronics 118, and possibly also, position-tracking system 24, and be designed to fit into very tight spaces.

As seen in FIG. 7E, radioactive-emission probe 22 may be formed as a multi-pixel probe, having a plurality of single-pixel radioactive-emission probes 40, of FIG. 1A, each having radiation detector 46 and tube collimator 48, wherein each collimator 48 may be pointing in a different direction.

As seen in FIG. 7F, intracorporeal-imaging head 100 may include a surgical instrument 105, preferably enclosed in a second catheter 110. Multi-lumen catheters are known in the art. Surgical instrument 105 may be a biopsy needle, a knife, a cryosurgery device, a resection wire, a laser ablation device, an ultrasound ablation device, other devices for localized radiation ablations, devices for implanting brachytherapy seeds, and other minimally invasive devices, as known.

As seen in FIGS. 7G-7H, radioactive-emission probe 22 may be formed as a multi-pixel probe 70, with detector pixels 72 arranged radially about a center, each pixel 72 having a collimator 76, wherein collimators 76 fan out, in a manner similar to flower petals. Alternatively, collimators 76 may be rectangular collimators. FIG. 7G provides a cross-sectional view of probe 70, and FIG. 7H provides a side view of intracorporeal-imaging head 100 and probe 70.

As seen in FIG. 7I intracorporeal-imaging head 100 may further include position tracking system 24.

FIGS. 7J-7K schematically illustrate intracorporeal-imaging head 1.00 moving for example, in the direction of an arrow 107, in a body lumen 109, wherein a malignant tumor 103, operative as radioactive-emission source 10, emitting radiation 12, maybe located.

Preferably, as seen in FIG. 7J, tumor 103 is first detected by camera 113, visually or by infrared vision.

Additionally, as seen in FIG. 7K, radioactive-emission source 10, emitting radiation 12, is then detected by radioactive-emission probe 22. Additionally, ultrasound and (or) MRI images of tumor 103 may also be obtained, by ultrasound transducer 108 (FIG. 7A) and MRI probe 116 (FIG. 7A).

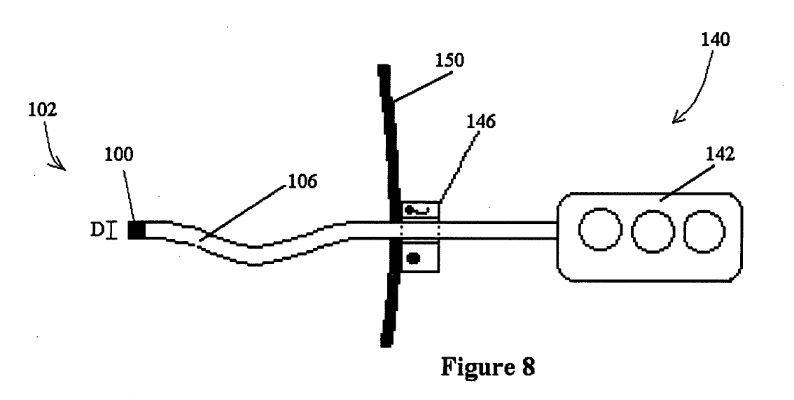

Referring further to the drawings, FIG. 8 illustrates an endoscope 140 for use in minimally invasive surgery. Endoscope 140 includes an extracorporeal portion having a control unit 142 and a catheter 106, adapted for insertion via a trucar valve 146, through a tissue 150. Intracorporeal-imaging head 100 may be mounted at distal end 102 of catheter 106.

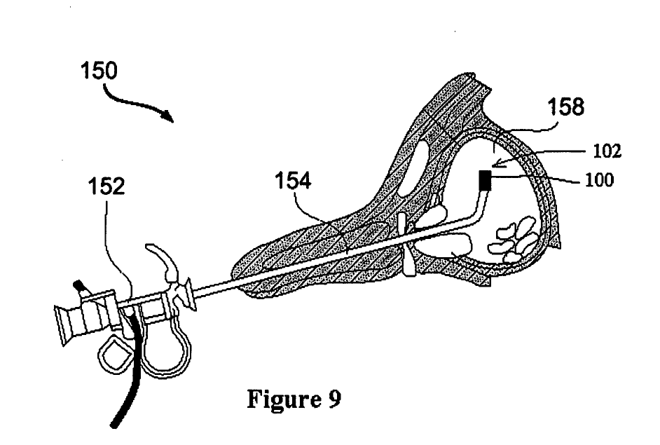

Referring further to the drawings, FIG. 9 illustrates a resectoscope 150, which includes an extracorporeal portion having a control unit 152 and an insertion tube 154, adapted for insertion to a bladder 158. Intracorporeal-imaging head 100 may be mounted at distal end 102 of catheter tube 154.

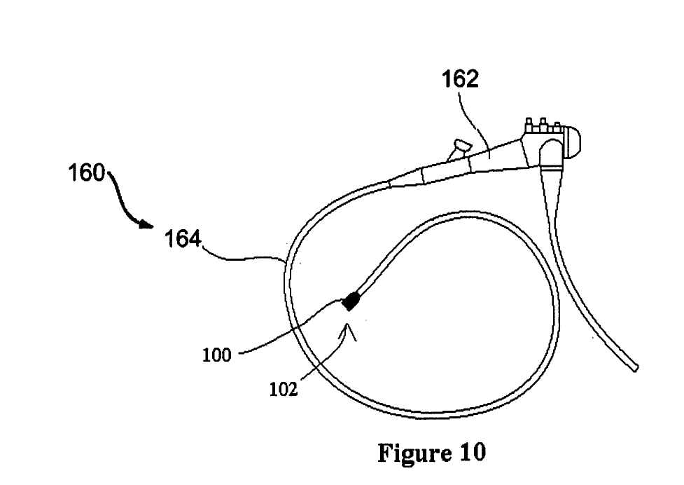

Referring further to the drawings, FIG. 10 illustrates an endoscope 160, which includes an extracorporeal portion having a control unit 162 and an insertion tube 164, adapted for insertion to a body lumen, such as the gastrointestinal track. Intracorporeal-imaging head 100 may be mounted at distal end 102 of catheter tube 164.

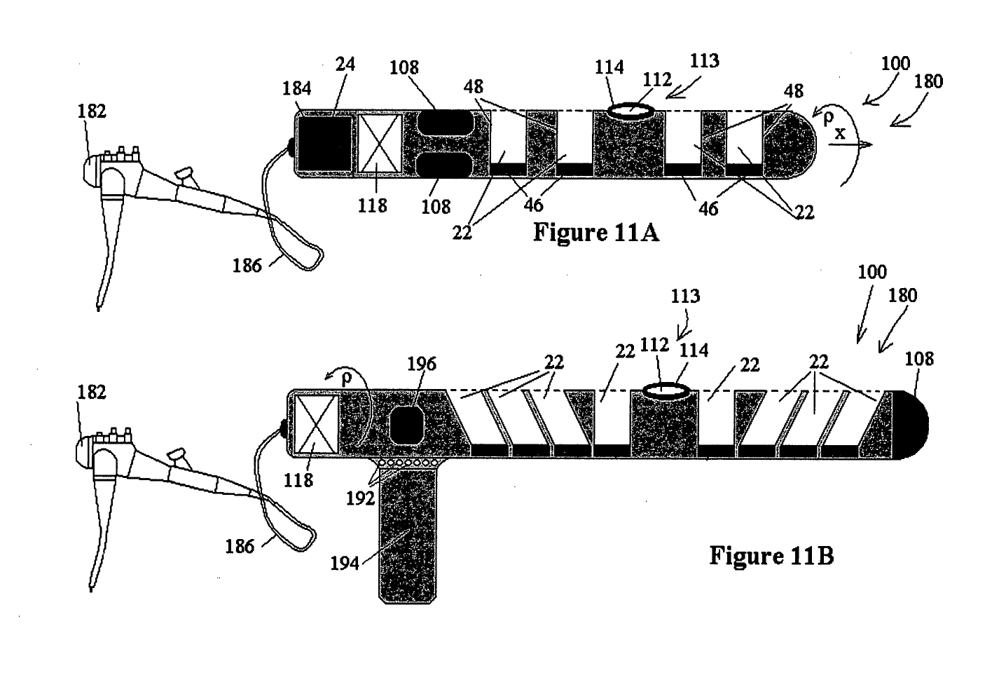

Referring further to the drawings, FIGS. 11A and 11B illustrate devices for rectal insertion, as colonoscopes, each including an extracorporeal portion, having a control unit 182 and an insertion tube 180, with intracorporeal-imaging head 100, as taught in conjunction with FIGS. 7A-7L hereinabove.

FIG. 11A illustrates an embodiment, which includes a motor 184, for example, a B-K motor, of B-K Medical A/S, of Gentofte, DK, for movement in the x direction and rotation ρ around the x axis. Additionally, motor 184 is adapted to report extracorporeally the exact position and orientation of intracorporeal-imaging head 100, based on the number of rotations from the point of entry. In this manner, motor 184 is operative as position tracking system 24. Additionally, intracorporeal-imaging head 100 may include camera 113, radioactive-emission probes 22, ultrasound detectors 108, and related electronics 118.

FIG. 11B illustrates an embodiment, in which a motor 196 provides a rotational motion only, in direction p, and is held in place by an arm 194, which rests against the groins and is in communication with intracorporeal-imaging head 100 via ball bearings 192. FIG. 11B further illustrates a different geometry of radioactive-emission probes 22.

It will be appreciated that many other geometries of intracorporeal-imaging head 100 are similarly possible.

It will be appreciated that the foregoing relates to detection as well as to imaging.

As used herein, detecting relates to performing instantaneous sensing, which may provide a “Yes” or “No” answer to the question, “Is there a suspicious finding?” Imaging, on the other hand, relates to constructing an image. Where desired, the image may be stored as a function of time to further construct a “movie” of the images.

Preferably, detecting is performed first, for example, as part of screening or regular checkup procedures, and imaging is performed as a follow-up, when the detection results call for it.

As used herein, the term about refers to ±20%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications in printed or electronic form, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An intracorporeal-imaging head, comprising:
a housing, which comprises:
a first optical imaging system, mounted on said housing, adapted to optically image a portion of a tissue; and
at least one radioactive-emission probe, mounted on said housing, adapted to image radioactive-emission from said portion.

2. The intracorporeal-imaging head of claim 1, comprising a position-tracking device, mounted on said housing, in a fixed positional relation with said radioactive-emission probe, for providing positional information for said radioactive-emission probe.

3. The intracorporeal-imaging head of claim 2, wherein said position-tracking device has six degrees of freedom.

4. The intracorporeal-imaging head of claim 2, adapted for obtaining high-resolution, radioactive-emission imaging by collimation-deconvolution algorithms.

5. The intracorporeal-imaging head of claim 1, wherein said first optical imaging system includes:
a lighting system, adapted to shine light on intracorporeal objects;
an lens, for focusing images of said intracorporeal objects; and
a light detecting array, for detecting said images of said intracorporeal objects.

6. The intracorporeal-imaging head of claim 1, comprising a second optical imaging system, adapted for zooming in on suspected pathologies, identified by said first optical imaging system.

7. The intracorporeal-imaging head of claim 6, wherein said second optical imaging system is a video camera.

8. The intracorporeal-imaging head of claim 6, wherein said second optical imaging system is a still camera.

9. The intracorporeal-imaging head of claim 1, wherein said radioactive-emission probe is a single-pixel probe.

10. The intracorporeal-imaging head of claim 1, wherein said radioactive-emission probe is a single-pixel, collimated probe.

11. The intracorporeal-imaging head of claim 10, wherein said single-pixel, collimated probe has a tube collimator.

12. The intracorporeal-imaging head of claim 10, wherein said single-pixel, collimated probe has a wide-angle collimator.

13. The intracorporeal-imaging head of claim 1, wherein said radioactive-emission probe is a multi-pixel probe.

14. The intracorporeal-imaging head of claim 1, wherein said radioactive-emission probe is a multi-pixel, collimated probe.

15. The intracorporeal-imaging head of claim 14, wherein said multi-pixel, collimated probe has tube collimators.

16. The intracorporeal-imaging head of claim 14, wherein said multi-pixel, collimated probe has wide-angle collimators.

17. The intracorporeal-imaging head of claim 1, wherein said housing is tubular, and said radioactive-emission probe is a multi-pixel probe, with detector pixels arranged radially about a center, each pixel having a collimator.

18. The intracorporeal-imaging head of claim 17, wherein said collimators are rectangular.

19. The intracorporeal-imaging head of claim 17, wherein said collimators fan out, in a manner similar to flower petals.

20. The intracorporeal-imaging head of claim 1, wherein said at least one radioactive-emission probe comprises a plurality of radioactive-emission probes.

21. The intracorporeal-imaging head of claim 1, comprising at least one ultrasound-imaging device.

22. The intracorporeal-imaging head of claim 1, comprising an MRI imaging device.

23. The intracorporeal-imaging head of claim 1, adapted for rotation.

24. The intracorporeal-imaging head of claim 1, adapted to be mounted on an endoscope for insertion through a trucar valve.

25. The intracorporeal-imaging head of claim 1, adapted to be mounted on an endoscope for insertion through a body lumen.

26. The intracorporeal-imaging head of claim 1, adapted to be mounted on a resectoscope for insertion through a urinary tract.

27. The intracorporeal-imaging head of claim 1, adapted to be mounted on a colonoscope.

28. The intracorporeal-imaging head of claim 1, comprising a surgical instrument.

29. The intracorporeal-imaging head of claim 1, wherein said first optical imaging system is a video camera.

30. The intracorporeal-imaging head of claim 1, wherein said first optical imaging system is a still camera.

31. A method of intracorporeal imaging, comprising:
providing an imager;
performing a first optical imaging of an intracorporeal portion of a tissue, by said imager; and
performing a radioactive-emission imaging of said portion, by said imager.

32. The method of claim 31, comprising performing said radioactive-emission imaging with a wide-aperture collimation probe, and position tracking said probe.

33. The method of claim 32, and further including obtaining high-resolution, radioactive-emission imaging by collimation-deconvolution algorithms.

34. The method of claim 31, wherein said performing said first optical imaging includes:
shining a light on intracorporeal objects;
focusing images of said intracorporeal objects; and
detecting said images of said intracorporeal objects.

35. The method of claim 31, comprising:
identifying suspected pathologies by a first optical imaging system; and
zooming in suspected pathologies by a second optical imaging system.

36. The method of claim 35, wherein said second optical imaging system is a video camera.

37. The method of claim 35, wherein said second optical imaging system is a still camera.

38. The method of claim 31, wherein said performing said radioactive-emission imaging includes performing said radioactive-emission imaging by a single-pixel radioactive-emission probe.

39. The method of claim 31, wherein said performing said radioactive-emission imaging includes performing said radioactive-emission imaging by a single-pixel, collimated probe.

40. The method of claim 39, wherein said single-pixel, collimated probe has a tube collimator.

41. The method of claim 39, wherein said single-pixel, collimated probe has a wide-angle collimator.

42. The method of claim 31, wherein said performing said radioactive-emission imaging includes performing said radioactive-emission imaging by a multi-pixel radioactive-emission probe.

43. The method of claim 31, wherein said performing said radioactive-emission imaging includes performing said radioactive-emission imaging by a multi-pixel, collimated probe.

44. The method of claim 43, wherein said multi-pixel, collimated probe has tube collimators.

45. The method of claim 43, wherein said multi-pixel, collimated probe has wide-angle collimators.

46. The method of claim 31, wherein said performing said radioactive-emission imaging includes performing said radioactive-emission imaging by a multi-pixel probe, with detector pixels arranged radially about a center, each pixel having a collimator.

47. The method of claim 46, wherein said collimators are rectangular.

48. The method of claim 46, wherein said collimators fan out, in a manner similar to flower petals.

49. The method of claim 31, wherein said performing said radioactive-emission imaging includes performing said radioactive-emission imaging by a plurality of radioactive-emission probes.

50. The method of claim 31, and further including imaging said portion by ultrasound.

51. The method of claim 31, and further including imaging said portion by MRI.

52. The method of claim 31, wherein said first optical imaging system is a video camera.

53. The method of claim 31, wherein said first optical imaging system is a still camera.

54. An intracorporeal-detecting head, comprising:
housing, which comprises:
a first optical detecting system, mounted on said housing, adapted to optically view a portion of a tissue; and
at least one radioactive-emission probe, mounted on said housing, adapted to detect radioactive-emission from said portion.

55. The intracorporeal-detecting head of claim 54, comprising a position-tracking device, mounted on said housing, in a fixed positional relation with said radioactive-emission probe, for providing positional information for said radioactive-emission probe.

56. The intracorporeal-detecting head of claim 55, wherein said position-tracking device has six degrees of freedom.

57. The intracorporeal-detecting head of claim 55, adapted for obtaining high-resolution, radioactive-emission detecting by collimation-deconvolution algorithms.

58. The intracorporeal-detecting head of claim 54, wherein said first optical detecting system includes:
a lighting system, adapted to shine light on intracorporeal objects;
an lens, for focusing instantaneous images of said intracorporeal objects; and
a light detecting array, for detecting said instantaneous images of said intracorporeal objects.

59. The intracorporeal-detecting head of claim 54, comprising a second optical detecting system, adapted for zooming in on suspected pathologies, identified by said first optical detecting system.

60. The intracorporeal-detecting head of claim 54, wherein said radioactive-emission probe is a single-pixel probe.

61. The intracorporeal-detecting head of claim 54, wherein said radioactive-emission probe is a single-pixel, collimated probe.

62. The intracorporeal-detecting head of claim 61, wherein said single-pixel, collimated probe has a tube collimator.

63. The intracorporeal-detecting head of claim 61, wherein said single-pixel, collimated probe has a wide-angle collimator.

64. The intracorporeal-detecting head of claim 54, wherein said radioactive-emission probe is a multi-pixel probe.

65. The intracorporeal-detecting head of claim 54, wherein said radioactive-emission probe is a multi-pixel, collimated probe.

66. The intracorporeal-detecting head of claim 65, wherein said multi-pixel, collimated probe has tube collimators.

67. The intracorporeal-detecting head of claim 65, wherein said multi-pixel, collimated probe has wide-angle collimators.

68. The intracorporeal-detecting head of claim 54, wherein said housing is tubular, and said radioactive-emission probe is a multi-pixel probe, with detector pixels arranged radially about a center, each pixel having a collimator.

69. The intracorporeal-detecting head of claim 68, wherein said collimators are rectangular.

70. The intracorporeal-detecting head of claim 68, wherein said collimators fan out, in a manner similar to flower petals.

71. The intracorporeal-detecting head of claim 54, wherein said at least one radioactive-emission probe comprises a plurality of radioactive-emission probes.

72. The intracorporeal-detecting head of claim 54, comprising at least one ultrasound-detecting device.

73. The intracorporeal-detecting head of claim 54, comprising an MRI detecting device.

74. The intracorporeal-detecting head of claim 54, adapted for rotation.

75. The intracorporeal-detecting head of claim 54, adapted to be mounted on an endoscope for insertion through a trucar valve.

76. The intracorporeal-detecting head of claim 54, adapted to be mounted on an endoscope for insertion through a body lumen.

77. The intracorporeal-detecting head of claim 54, adapted to be mounted on a resectoscope for insertion through a urinary tract.

78. The intracorporeal-detecting head of claim 54, adapted to be mounted on a colonoscope.

79. The intracorporeal-detecting head of claim 54, comprising a surgical instrument.

80. A method of intracorporeal detecting, comprising:
providing an detector;
performing a first optical detecting of an intracorporeal portion of a tissue, by said detector; and
performing a radioactive-emission detecting of said portion, by said detector.
